(19) United States
(12) Patent Application Publication
(10) Pub. No.: US 2007/0120041 A1
(43) Pub. Date: May 31, 2007

(54) Sealed Package With Glass Window for Optoelectronic Components, and Assemblies Incorporating the Same

(76) Inventors: Lior Shiv, Hilleroed (DK); Jochen Kuhmann, Berlin (DE)

Correspondence Address: FISH & RICHARDSON P.C., P.O. BOX 1022, MINNEAPOLIS, MN, 55440-1022, US

(21) Appl. No.: 11/558,175

(22) Filed: Nov. 9, 2006

Related U.S. Application Data
(60) Provisional application No. 60/735,485, filed on Nov. 10, 2005.
(60) Provisional application No. 60/737,532, filed on Nov. 15, 2005.
(60) Provisional application No. 60/749,247, filed on Dec. 9, 2005.

(51) Int. Cl. C12Q 1/68; H01L 23/02

(52) U.S. Cl. 250/200; 257/680

(57) ABSTRACT

A package includes one or more optoelectronic components and a cap with an embedded glass window attached to a substrate. The optoelectronic component(s) is supported by the substrate and is capable of detecting or emitting light through the glass window. The glass window may serve as an optical filter. Techniques are disclosed for fabricating a relatively thin package with an embedded glass window in the cap.

### CROSS-REFERENCE TO RELATED APPLICATIONS

This disclosure claims the benefit of U.S. provisional applications nos. 60/735,485, filed Nov. 10, 2005; 60/737,532, filed Nov. 15, 2005; and 60/749,247, filed Dec. 9, 2005. The disclosures of the provisional applications are incorporated herein by reference.

### BACKGROUND

The present disclosure relates to packaging for optoelectronic components.

Proper packaging of optoelectronic components is important to ensure the integrity of the signals to and from the micro components and often determines the overall cost of the assembly.

Packaging for optoelectronic components also needs to include a way for the light signals to enter or exit the package.

U.S. Pat. No. 6,818,464, assigned to the assignee of this application, discloses a technique for fabricating a package that can be used to house, for example, an optoelectronic component. As disclosed in that patent, the optoelectronic component may be mounted to a base. The base includes an optical waveguide formed along its surface, with the optoelectronic component coupled to the waveguide. A semiconductor cap can be attached to the base so as to hermetically enclose the optoelectronic component. However, use of such an optical waveguide may not be particularly suited for some applications.

### SUMMARY

A package is disclosed for housing one or more optoelectronic components. In addition, techniques are disclosed for fabricating a relatively thin package that houses one or more optoelectronic components. The package may be fabricated, for example, in a wafer-level batch process and includes a glass window embedded in a cap structure to allow light signals from outside the package to be detected by the optoelectronic component housed within the package or to allow a light signal generated by the optoelectronic component to be emitted from the package.

In one aspect, the package includes an optoelectronic component, a substrate with a front surface supporting the optoelectronic component, and a cap including an embedded glass window attached to the substrate. The cap and the substrate define an interior region that encloses the optoelectronic component and the optoelectronic component is positioned to detect or emit light through the glass window.

In various implementations, one or more of the following features may be present. For example, the glass window may be adapted to function as an optical filter. In one implementation, a film may be deposited on at least one side of the glass window to reflect at least one predetermined wavelength of light. In another implementation, the glass window may have one or more color pigments to selectively absorb at least one predetermined wavelength of light.

The package may also include one or more feed-through interconnects through the cap to electrically couple the optoelectronic component to a contact on the exterior of the package. The feed-through interconnects may or may not be hermetically sealed.

The glass window may be embedded in a semiconductor material. In addition, the glass window may be formed of a material having a thermal coefficient that matches the thermal coefficient of the semiconductor material.

The package can also be incorporated as part of an assembly that includes, for example, a lens barrel.

In another aspect, a method for fabricating a package includes attaching a cap having an embedded glass window to a substrate having a front surface that supports an optoelectronic component so that the cap and substrate define an interior region that encloses the optoelectronic component. The optoelectronic component is positioned to detect or emit light through the glass window.

The glass window may be embedded in the cap by forming a cavity in a surface of a semiconductor material and depositing glass in the cavity. According to one implementation, glass particles may be deposited and caused to settle in the cavity, and the glass particles may be melted to form the glass window. The back surface of the cap may be thinned until, for example, a back surface of the glass window is exposed. In another implementation, the thinning may be stopped when the cap is of a predetermined thickness. In addition, the glass window may be polished. The thinning and polishing of the back surface of the cap may also be done after attaching the cap to the substrate.

The details of one or more implementations are set forth in the accompanying drawings and the description below. Other features and advantages will be apparent from the description and drawings, and from the claims.

### BRIEF DESCRIPTION OF DRAWINGS

FIG. 1 illustrates a cross-sectional view of a package housing one or more optoelectronic components according to an implementation of the present invention.

FIGS. 2-4 illustrate steps in an example of a fabrication process of a semiconductor wafer for a cap structure with a glass window according to the invention.

FIG. 5 illustrates the semiconductor wafer for the cap structure with the glass window bonded to a second wafer in which one or more optoelectronic components are processed or on which they are mounted.

FIG. 6 illustrates the wafers of FIG. 5 after thinning the back-side of the wafer for the cap structure.

FIG. 7 illustrates a dicing process to separate individual packages from one another.

FIGS. 8 and 9 illustrate examples of assemblies that incorporate a lens barrel and package according to the invention.

### DETAILED DESCRIPTION

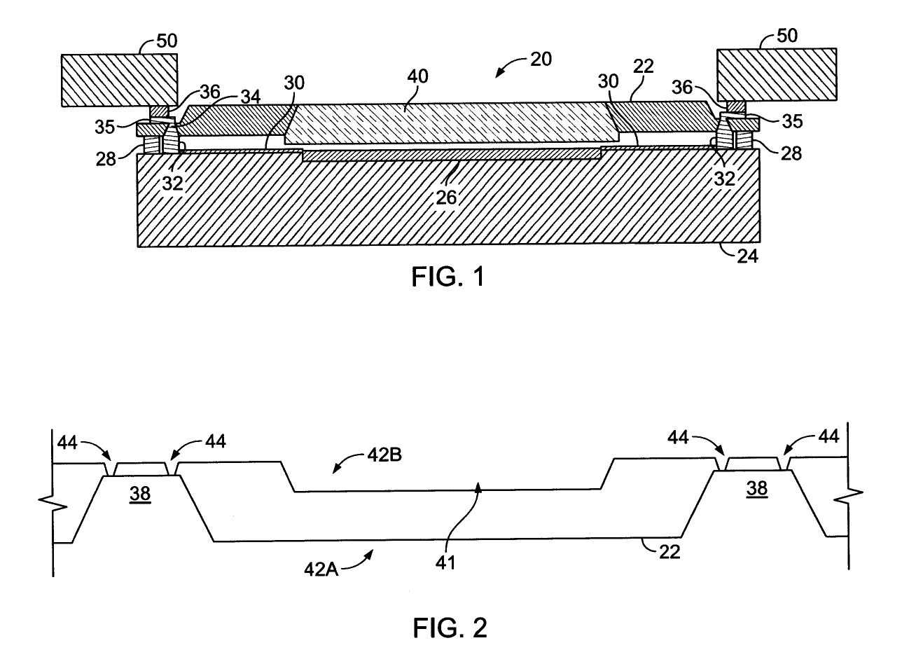

As shown in FIG. 1, a package 20 includes a cap 22 and a substrate (or base) 24.

The base 24 may comprise, for example, a semiconductor material, such as silicon, or a glass material. One or more optoelectronic components 26 (e.g., light receiving or emitting devices or optoelectronic integrated chips) are mounted to, or integrated with, the base 24, which may be bonded to the cap 22, for example, by a sealing ring 28.

The cap 22 may comprise, for example, a semiconductor material such as silicon and includes a glass window 40 embedded within the silicon. The window 40 is located opposite the active area of the optoelectronic component 26, which may be hermetically sealed within the package.

The glass window 40 embedded in the cap structure also may serve as an optical filter. For example, a thin film or other coating may be deposited on one or both sides of the glass to limit the wavelength(s) of light that can be transmitted through the glass window 40. Such films or coatings may reflect, for example, infra-red light or other ranges of the optical spectrum. Alternatively, color pigments may be incorporated into the glass so that certain wavelengths are absorbed and, thus, not transmitted through the glass window.

In a particular application, the package may house, for example, one or more charge-coupled devices (CCDs) as part of a digital image sensor.

Electrically conductive lines 30 may extend along the surface of the base 24 from the components 26 to electrically conductive bumps 32 that are electrically connected to feed-through metallization 34 extending through micro-vias in the cap 22. At the exterior surface of the cap 22, the feed-through metallization serves as surface mount pads 35 which, in turn, may be electrically coupled to solder bumps 36. The solder bumps 36 may be connected, for example, to a printed circuit board 50. Using the techniques described in this disclosure, the final thickness of the cap 22 may be made as small as 200 μm or less for some implementations.

Multiple packages may be fabricated simultaneously in a wafer-level batch process. For example, multiple cap structures may be fabricated on a first wafer (which may be referred to as a “cap-wafer”). The cap-wafer then may be bonded to a second wafer (which may be referred to as a “device-wafer”) on which optoelectronic components 26 are mounted. The device-wafer may serve as a substrate that forms the bases of the packages.

As explained in greater detail below, the cap-wafer may have an initial thickness, for example, on the order of several hundred microns (e.g., 300-700 μm). The wafer may have a diameter, for example, of four inches. Larger diameter (e.g., 6-inch) wafers also may be suitable for some implementations. After the cap-wafer is bonded to the device-wafer, a mechanical grinding or other process may be used to thin the back-side of the cap-wafer so that the resulting caps have a desired thickness, which may be as small as 200 μm or less. The wafers subsequently can be provided with solder-bumps, reflown, and diced to form individual packages housing the optoelectronic component(s).

One process which may be used to fabricate multiple cap structures on a wafer employs a double-sided etching technique. As shown in FIG. 2, the double-sided etching technique may be used to form cavities 38 on the back-side 42A of the cap-wafer. The cavities serve as the boundary between adjacent cap-structures.

During the double-sided etch process, micro-vias 44 for the feed-through metallization may be etched from the front-side 42B of the cap-wafer. Preferably, the micro-vias 44 are formed near the edges of the cavities 38. In addition, a relatively deep cavity 41 (e.g., 100-200 μm) is formed in the front-side 42B. The cavity 41 serves as a mold for the glass window 40 subsequently formed in the cap-wafer. Various etching techniques may be used to form the cavities 38, 41 and micro-vias 44 depending on the material of the cap-wafer.

A wafer that is suitable for forming the caps 22 may have, for example, a multi-layer structure that includes a substantially etch-resistant layer sandwiched between first and second semiconductor layers. The first and second semiconductor layers may include, for example, silicon, and the etch-resistant layer may include, for example, silicon nitride, silicon oxy-nitride or silicon dioxide. One suitable etching technique uses a KOH wet etch. Further details of a multi-layer structure and examples of etching techniques are disclosed in U.S. Pat. No. 6,818,464, mentioned above. The disclosure of that patent is incorporated herein by reference. Other wafer structures and other etching techniques may be used as well. For example, although FIG. 2 shows the sidewalls of the cavities 38, 41 as being sloped, other etching techniques may result in sidewalls that are substantially vertical.

As can be seen from the example of FIG. 2, following formation of the cavities 38 and micro-vias 44, the cap-wafer still may have an overall thickness on the order of several hundred microns (e.g., 300-700 μm). Such a thickness facilitates subsequent handling and processing of the cap-wafer and reduces the likelihood of damage that might occur if the wafer were thinner.

After formation of the cavities 38, 41 and micro-vias 44, the glass window 40 is formed by depositing glass in the cavity 41. Preferably, the glass window 40 should be formed of a material that matches the thermal coefficient of expansion of the material that forms the cap 22. In addition, the glass window 40 should be transparent to the wavelength(s) of light that the optoelectronic component is designed to emit or detect.

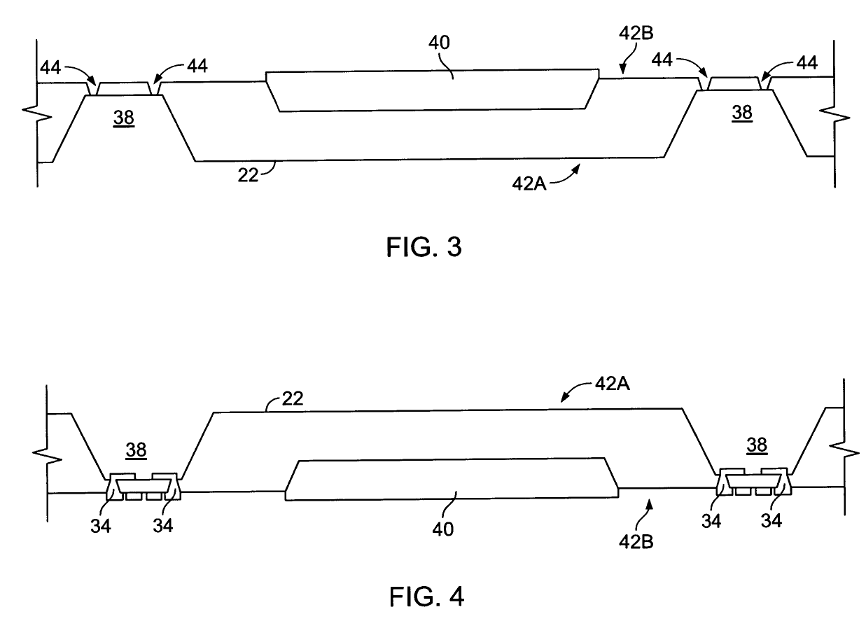

Various techniques may be used to deposit the glass in the cavity 41. For example, a dispenser or stencil print can be used. Alternatively, small balls of glass may be deposited on the cap-wafer, which then is vibrated until the glass balls fall into the cavities 41. After firing the glass, mechanical grinding and polishing processes may be performed to smooth the surface of the glass window 40 (see FIG. 3). Preferably, the grinding and polishing should stop before reaching the front-side 42B of the cap-wafer. After the glass is deposited in the cavity 41, the glass window 40 and the wafer may be molded together or may form an integral unit.

Next, the micro-vias 44 may be hermetically sealed (see FIG. 4), for example, using an electroplated feed-through metallization technique. The feed-through metallization 34 also may include a diffusion barrier, and the sealing material may include, for example, a non-noble metal. Further details of such feed-through metallization techniques are disclosed in U.S. Pat. No. 6,818,464, previously mentioned.

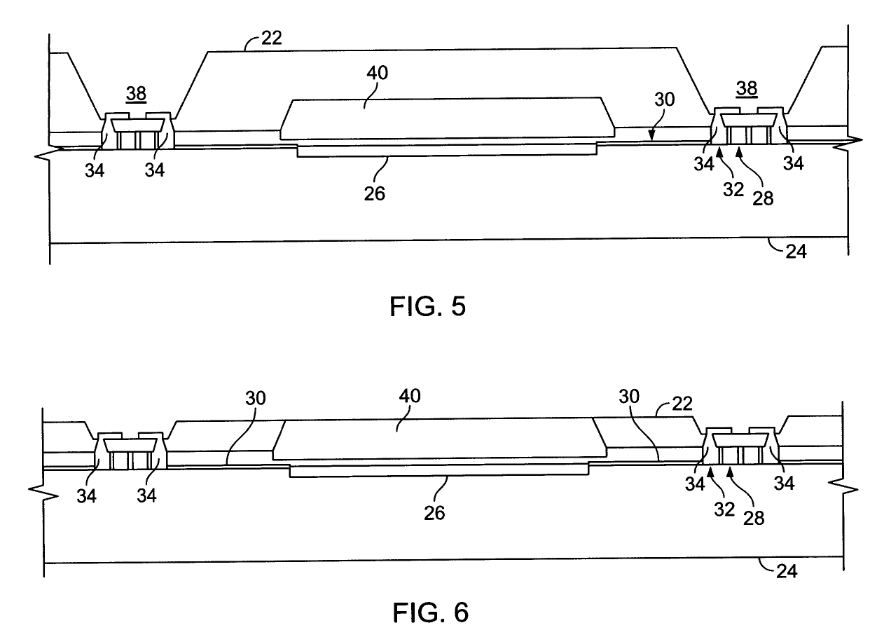

The electrically conductive bumps 32 may be provided on the front-side 42B of the cap-wafer in electrical contact with the feed-through metallization 34 (see FIG. 5).

The cap-wafer then may be bonded to the device-wafer that serves as the substrate on which the optoelectronic components 26 are mounted. The cap-wafer and device-wafer are aligned so that the electrically conductive bumps 32 contact the electrically conductive lines 30 extending along the surface of the base 24 from the optoelectronic component 26 which fits within the area between the wafers. As discussed above, a sealing ring 28 may provide a seal so that the component 26 is hermetically housed in the area between the wafers.

After the cap-wafer and device-wafer are bonded, for example, as shown in FIG. 5, the back-side of the cap-wafer (including the glass window 40) is thinned to a desired thickness as illustrated, for example, in FIG. 6. Various techniques may be used for the thinning process, including mechanical grinding or polishing techniques. Performing thinning of the cap-wafer after it is bonded to the device-wafer, rather than beforehand, may reduce the likelihood that damage will occur during subsequent handling of the thin cap-wafer.

The amount of thinning will vary depending on the particular application. However, the extent of the thinning may be significant and, in some implementations, may be on the order of 50 μm to several hundred microns. Thus, the final thickness of the cap-wafer for some implementations may be in the range of about 30-70% of the initial wafer thickness. The cap-wafer may be thinned to a final thickness as small as 200 μm or less.

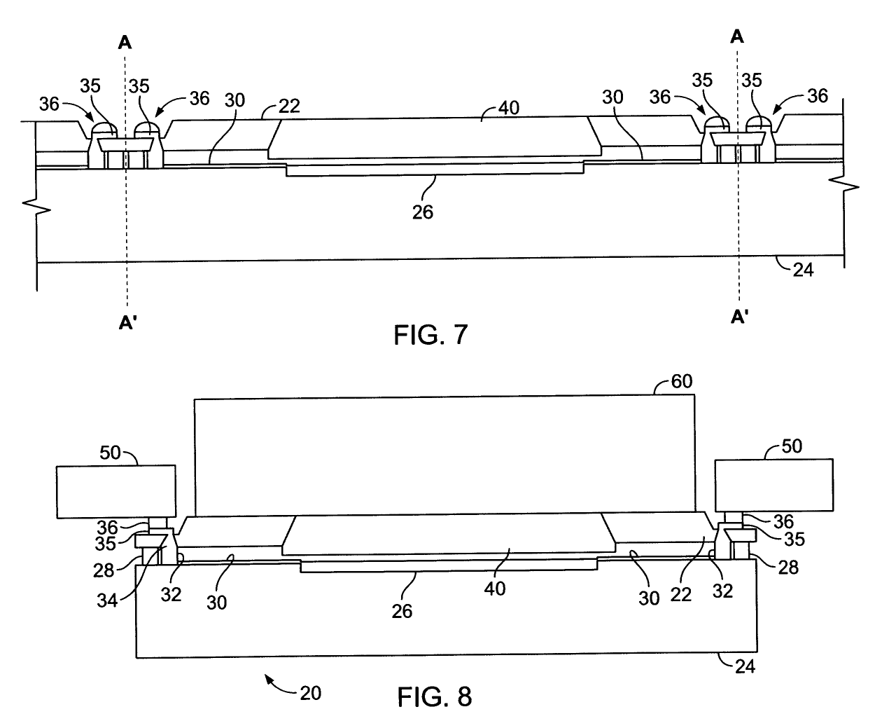

After thinning the back-side of the cap-wafer, a screen printing or other process may be performed to provide the solder bumps 36 on the back-side pads 35 (see FIG. 7).

The wafers then can be diced, for example along lines A-A′, to form individual packages each of which houses one or more optoelectronic components 26.

The foregoing techniques can provide a relatively thin package that includes hermetically sealed feed-through electrical connections coupling the optoelectronic component to electrical contacts on an exterior surface of the package.

In the foregoing implementations, mirco-vias 44 that extend from the front-side 42B of the cap-wafer to the back-side 42A may be formed in the cap structure 22 before providing the feed-through metallization 34. In other implementations, micro-vias 44 for the feed-through metallization 34 need not extend completely through the wafer before providing the feed-through metallization 34. For example, micro-vias 44 extending only partially through the wafer may be formed, and then feed-through metallization 34 may be provided in the micro-vias 44. During the subsequent back-side wafer-thinning process, the feed-through metallization 34 is exposed so that electrical contacts to the feed-through metallization 34 may be provided.

Other micro-components may be integrated into the package.

The optoelectronic components 26 housed within the package are housed within an area defined by the first and second wafers (e.g., the cap-wafer and the device-wafer). They may be mounted on one of the wafers or they may be integrated within one of the wafers.

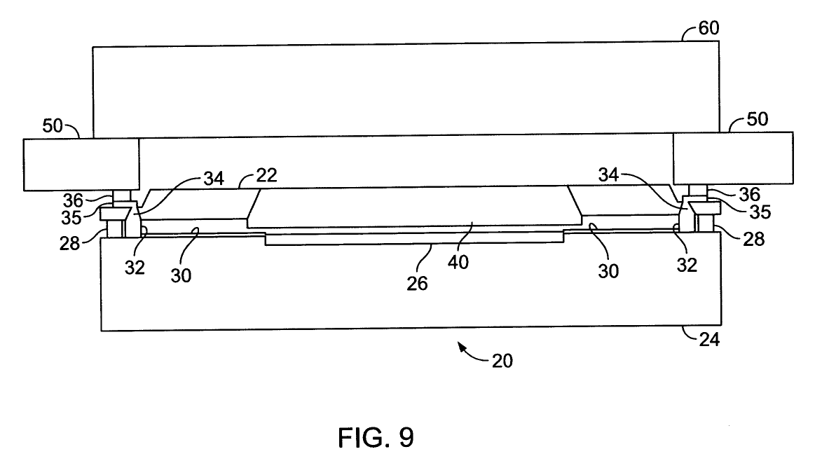

FIGS. 8 and 9 illustrate examples of assemblies that incorporate a lens barrel 60 and also a package 20 for optoelectronic components (e.g., a CMOS image sensor) as described above.

A number of implementations have been described. Various modifications may be made without departing from the spirit and scope of the invention. Accordingly, other implementations are within the scope of the claims.

What is claimed is:

1. A package comprising:
an optoelectronic component;
a substrate with a front surface supporting the optoelectronic component; and
a cap comprising an embedded glass window,
wherein the cap is attached to the substrate to define an interior region that encloses the optoelectronic component, and
wherein the optoelectronic component is positioned to detect or emit light through the glass window.

2. The package of claim 1 wherein the glass window is adapted to function as an optical filter.

3. The package of claim 1 further comprising a film deposited on at least one side of the glass window to reflect at least one predetermined wavelength of light.

4. The package of claim 1 further comprising one or more color pigments in the glass window to selectively absorb at least one predetermined wavelength of light.

5. The package of claim 1 including one or more feed-through interconnects through the cap for electrically coupling the optoelectronic component to a contact on the exterior of the package.

6. The package of claim 5 wherein the one or more feed-through interconnects is hermetically sealed.

7. The package of claim 1 wherein the cap and the substrate form a hermetic seal.

8. The package of claim 1 wherein the window is embedded in a semiconductor material.

9. The package of claim 8 wherein the material of the window has a thermal coefficient that substantially matches the thermal coefficient of the semiconductor material.

10. A method comprising:
providing a cap having an embedded glass window; and
attaching the cap to a substrate having a front surface that supports an optoelectronic component,
wherein the cap and substrate define an interior region that encloses the optoelectronic component which is positioned to detect or emit light through the glass window.

11. The method of claim 10 wherein providing a cap having an embedded glass window includes:
forming a cavity in a surface of a semiconductor material; and
depositing glass in the cavity to form a window embedded in the semiconductor material.

12. The method of claim 10 wherein providing a cap having an embedded glass window includes thinning a back surface of the cap.

13. The method of claim 12 including stopping the thinning after a back surface of the glass window is exposed.

14. The method of claim 12 including stopping the thinning when the thickness of the cap reaches a predetermined value.

15. The method of claim 10 wherein providing a cap having an embedded glass window includes polishing a front surface of the glass window.

16. The method of claim 10 wherein providing a cap having an embedded glass window includes polishing a back surface of the glass window.

17. The method of claim 10 wherein providing a cap having an embedded glass window includes depositing particles of glass in a cavity in a semiconductor material;
causing the particles of glass to settle in the cavity; and
melting the particles of glass to form the glass window.

18. The method of claim 10 wherein providing a cap having an embedded glass window includes forming a cavity in a surface of a semiconductor material;
depositing particles of glass in the cavity;
causing the particles of glass to settle in the cavity;
melting the particles of glass to form the glass window;
polishing a front surface of the glass window;
thinning a back surface of the semiconductor material; and
stopping the thinning when a back surface of the glass window is exposed or the thickness of the cap reaches a predetermined value.

19. The method of claim 10 further comprising thinning a back surface of the cap after attaching the cap to the substrate.

20. The method of claim 19 including stopping the thinning after a back surface of the glass window is exposed.

21. The method of claim 19 including stopping the thinning when the thickness of the cap reaches a predetermined value.

22. The method of claim 19 including polishing a back surface of the glass window.

23. The method of claim 10 including depositing a film on at least one side of the glass window to reflect at least one predetermined wavelength of light.

24. The method of claim 10 wherein the glass has one or more color pigments to selectively absorb at least one predetermined wavelength of light.

25. A method for providing a cap having an embedded glass window comprising:
forming a cavity in a surface of a semiconductor material; and
depositing glass in the cavity to form a glass window embedded in the semiconductor material.

26. The method of claim 25 including thinning a back surface of the semiconductor material.

27. The method of claim 26 including stopping the thinning when a back surface of the glass window is exposed.

28. The method of claim 26 including stopping the thinning when the thickness of the semiconductor material reaches a predetermined value.

29. The method of claim 25 wherein depositing glass in the cavity to form a glass window embedded in the semiconductor material includes:
depositing particles of glass in the cavity;
causing the particles of glass to settle in the cavity; and
melting the particles of glass to form the glass window.

30. The method of claim 25 including depositing a film on at least one side of the glass window to reflect at least one predetermined wavelength of light.

31. The method of claim 25 wherein the glass window has one or more color pigments to selectively absorb at least one predetermined wavelength of light.

32. The method of any one of claims 10 through 21 wherein the method is performed as a wafer-level process.
